(19) United States
(12) Patent Application Publication
(10) Pub. No.: US 2003/0224072 A1
(43) Pub. Date: Dec. 4, 2003

(54) Topical and oral administration of a composition containing Pelargonium Graveolens for diagnosis and treatment of various neuralgias and other conditions

(76) Inventor: Bruce Marshall Frome, Beverly Hills, CA (US)

Correspondence Address: Bruce M. Frome, PO Box 15157, Beverly Hills, CA, 90209, US

(21) Appl. No.: 10/446,754

(22) Filed: May 28, 2003

Related U.S. Application Data
(60) Provisional application No. 60/385,081, filed on Jun. 3, 2002.

(51) Int. Cl. A61K 35/78

(52) U.S. Cl. 424/736; 424/778; 424/742; 424/745; 424/764; 424/769

(57) ABSTRACT

This present invention describes a method of diagnosing and treating neuropathic pain, Bell's palsy, viral sore throat, pain due to burns, including sunburn, and pain and itching due to insect bites with compositions of essential aromatic oils, all of which contain Pelargonium graveolens (also called geranium oil bourbon or GOB). This invention discloses compositions of one or more of five other essential aromatic oils, particularly lavender, bergamot, blue chamomile, eucalyptus and tea tree, which when added to GOB acts synergistically with the GOB. These compositions can be used alone or in a composition with a pharmaceutically acceptable carrier in topical or oral dosage form. The present invention is an improvement in the methods and expansion of uses as stated in the previous United States Patent issued in 1993 entitled “Diagnosis and Treatment of Various Neuralgias” (Frome)—U.S. Pat. No. 5,260,313.

### CROSS-REFERENCE TO RELATED APPLICATION

This non-provisional utility application claims the benefits of the earlier provisional application No. 60/385,081 filed on Jun. 3, 2002. The present invention is a) an improvement in the methods as stated in the previous United States Patent issued in 1993 entitled “Diagnosis and Treatment of Various Neuralgias” (Frome)—U.S. Pat. No. 5,260,313 and b) by improving the method as stated in U.S. Pat. No. 5,260,313, the present invention expands the uses of the invention as stated in U.S. Pat. No. 5,260,313. The method of the present invention differs from the method of the invention described in U.S. Pat. No. 5,260,313 in that this invention is a composition of two or more aromatic essential oils as the active therapeutic or diagnostic agent while the invention detailed in U.S. Pat. No. 5,260,313 utilizes a single aromatic essential oil, specifically Pelargonium Graveolens, as the active therapeutic or diagnostic agent.

### BACKGROUND OF THE INVENTION

This invention relates to the topical and oral administration of various compositions of essential aromatic oils, all of which contain Pelargonium Graveolens, also called geranium oil bourbon and hereinafter sometimes referred to as GOB. This invention is used for diagnosis and treatment of pain that is neuropathic (nerve tissue injury) in origin and treatment of other miscellaneous conditions such as burns, viral sore throat, insect bites and Bell's palsy.

Neuropathic pain encompasses various pain syndromes associated with disorders due to injuries of the peripheral, sympathetic and central nervous system. Not only are curative measures for these neural abnormalities generally unavailable but symptomatic treatments are often ineffective causing an enormous problem for clinicians in treatment and management of neuropathic pain. Distinguishing neuropathic pain from somatic or visceral pain is often difficult.

This invention will not relieve somatic or visceral pain such as pain experienced due to arthritis. However, if pain relief does occur with use of this invention when applied topically, the diagnosis of neuropathic pain can be made with certainty in a non-bum subject. There are six million Americans suffering from various types of neuropathic pain syndromes, including sympathetically mediated pain such as reflex sympathetic dystrophy, peripheral neuropathy, post-herpetic neuralgia and tic doloreux. Neuropathic pain is the most common cause of suicide due to pain as narcotics and other analgesics are generally ineffective in relieving neuropathic pain.

Although pain from burns is not classified as neuropathic, the present invention was found to be highly effective in relieving pain due to burns after topical application to the burn site (including sunburn). Sore throat, accompanied by viral lesions (cold sores) of the lips, was temporarily or permanently eliminated after gargling with the present invention. Pain from some neuralgias (neuropathic pain), specifically post-herpetic neuralgia, sciatica, or reflex sympathetic dystrophy was partially or completely eliminated, although temporarily, after oral administration of the invention. The paresis accompanying Bell's palsy was substantially eliminated after oral administration or topical application of the invention to the affected side of the face if treated in the first three months after onset of the condition.

A. Neuropathic Pain Management

Neuropathic pain is caused by nerve tissue damage. This nerve damage may occur in a peripheral nerve such as found in diabetic neuropathy or stump pain in an amputee, or it may be sympathetically mediated as in reflex sympathetic dystrophy, or it may be thalamic in origin, for example, in a person who has suffered a stroke.

The types of pain associated with nerve injury are usually defined by their unique subjective effects such as allodynia, hyperpathia, hyperesthesia, hyperalgesia, dysesthesia parathesia, deafferentation pain and anesthesia delorosa The treatment of pain due to nerve injury (neuropathic pain) is directed towards relief of these subjective symptoms.

There are generally five categories of pain treatments due to nerve injury that are available:

1) Topical Treatments

a) Geranium Oil bourbon (GOB) (Applicant's U.S. Pat. No. 5,260,313).

The pain relief is often limited to a few hours or less and because of the full concentration of GOB, it exhibits a pungent odor which often discourages frequent use.

b) Ketamine and other NMDA receptor antagonists anticholergic agents and sympathetic blocking agents administered topically (Applicant's U.S. Pat. No. 6,197,830B and Applicant's U.S. Pat. No. 6,387,957B1).

These are all FDA controlled agents, which strongly suggests their use will be highly regulated by various government agencies, making their availability more difficult.

c) Capsaicin (cayenne pepper extract).

This agent, when applied to the skin, causes a burning sensation that continues for approximately one week until all of the substance P has been depleted from the area being tested. Capsaicin provides pain relief for only a small percentage of neuropathic pain patients. Furthermore, the initial burning sensation discourages its use as its full value does not take effect for approximately the one week stated and patients frequently abandon its use due to the burning sensation.

d) Topical local anesthetics.

Pain relief is short and minimal, occurring in less than 25% of neuropathic pain patients.

e) Topical salicylates.

Pain relief is also short and minimal, occurring in less than 10% of neuropathic pain patients.

2) Oral Medication. These include:

a) Standard analgesic medications such as NSAIDS and narcotics

b) Adrenergic blocking agents such as phenoxybenzamine

c) Anti-convulsants such as carbamazepine

d) Adrenergic blocking agents such as terazosine, and

e) Tricylic antidepressants such as amitriptylline

All of these oral medications provide minimal relief in the treatment of neuropathic pain with a great many side effects, which very frequently discourage their use.

3) Nerve Blocks

a) Stellate ganglion sympathetic—invasive procedure in medical facility

b) Lumbar sympathetic—invasive procedure in medical facility

c) Cervical sympathetic—invasive procedure in medical facility

d) Epidural nerve blocks, which are invasive procedures usually performed in a hospital by a physician specialist

e) Nerve sclerosing agents which are highly risky, often resulting in neuromas and are ineffective in the long term.

4) Surgical Procedures

a) Implantation of dorsal column stimulators and subarachnoid infusion pumps again must be performed by a physician in a hospital setting with many side effects and complications possible

b) Severing of the spinal cord or of nerves have been tried in the past resulting in greater pain than was originally present in the long term. This method presently has been abandoned.

5) Other Methods

Other seldom used methods such as hypnosis and deep brain stimulation have been tried and are rarely successful. Acupuncture, biofeedback and physical therapy also have been used with minor and short-term success for neuropathic pain relief.

B. Relief of Pain Due to Burns and Relief of Itching or Pain Due to Insect Bites

Pain or itching as a result of burns or insect bites, including sunburn is responsive to narcotics and other common analgesics which differs from neuropathic pain. However, in the instance of sunburn or insect bites, most people prefer a topical agent over oral medication as the topical agent relieves the pain or itching almost instantaneously versus a delayed onset with the oral medications, and the topical agent does not cause side effects often associated with the oral medications.

This invention is a treatment not previously available for burns, including sunburn and insect bites. The current available topical relief of pain or itching due to burns or insect bites containing local anesthetics generally lasts for a few minutes to two hours while this invention offers relief usually in excess of three hours and often in excess of 24 hours.

C. Treatment of Viral Sore Throat

Pain due to viral sore throat is often relieved after gargling with local anesthetics such as lidocaine or melting a lozenge with anesthetic in one's mouth. This invention is an alternative to the stated local anesthetic gargle or lozenge method. This invention relieves the pain of viral sore throat when gargling, especially if the sore throat is associated with herpetic viral lesions about the mouth or lips.

D. Treatment of Bell's Palsy

Bell's palsy, a motor disturbance of the facial muscles, is a unilateral facial paralysis of sudden onset and unknown cause. The pathology is assumed to be due to swelling of the facial cranial nerve due to viral infection with ischemia and compression of the facial nerve in the narrow confines of its course through the temporal bone. All present treatment, although frequently used, such as oral corticsteroids and antivirals are virtually ineffective. However, complete recovery within several months almost always follows partial paralysis. If total paralysis exists, complete recovery is rare.

This invention often cures Bell's palsy when the invention is swallowed or applied topically to the course of the affected facial cranial nerve in the initial stages of the paralysis, preferably in the first two weeks, thereby markedly shortening the cause of the paralysis. The invention is virtually ineffective if administered after three months of onset of the condition. The therapeutic mechanism is unclear but as in the relief of viral sore throat, it may be antiviral or the mechanism may be just a reduction of swelling of the affected facial nerve.

### REFERENCES CITED

U.S. Pat. Documents

| | | | |
|---|---|---|---|
| 4,311,617 | January 1982 | Ansari | 252/522R |
| 4,599,677 | July 1986 | Lawless | 361/321 |
| 4,923,685 | May 1990 | Wuelkhitz | 424/54 |
| 4,940,483 | July 1990 | Thompson | 424/195.1 |
| 5,260,313 | November 1993 | Frome | 514/552 |
| 6,197,830 | March 2001 | Frome | 514/183 |
| 6,387,957 | May 2002 | Frome | 514/647 |

Other Publications:

Embase Abstract, AN. 96229589, Lipman, A. G., “Analgesic drugs for neuropathic and sympathetically maintained pain”, Clinics in Geriatric Medicine, 12:501-515 (1996).

Gordon L. Flynn, General Intro. Topical and Transdermal Drug Delivery Systems Topical Drug Bioavailability, Bioequivalence, and Penetration, Plenum Press, 1993 Chapter 20, pp. 369-391.

O. Shimoda; T. Kano; Takaki, et. al., “Transdermal application of 10% Lidocaine-gel for management of pain associated with herpes zoster”: Department of Anesthesiology, Kumamoto Rosai Hospital, Yatsushiro. Masui August 1993; 42(8):1171-6, Abstract.

### BRIEF SUMMARY OF THE INVENTION

The present invention relates to compositions

a) used in a topical application for relief and diagnosis of neuropathic pain accompanied by a reduction of swelling and redness, if present, of the affected area;

b) for substantial elimination of pain due to viral sore throat (pharyngitis) when gargled;

c) for substantial elimination of facial paralysis in individuals suffering from Bell's palsy, when applied topically or administered orally, and if treated early in the course of the paralysis;

d) for substantial relief of pain due to burns of the skin (including sunburn) and for relief of pain and itching caused by insect bites (such as bee stings or mosquito bites); and

e) for substantial temporary relief of pain due to sciatica or post-herpetic neuralgia when administered orally.

The object of this invention is to provide a composition of essential aromatic oils that offers superior pain relief, or reduction in redness and swelling (if present) and is generally more effective than that achieved utilizing GOB as the sole agent as described in U.S. Pat. No. 5,260,313. This will result in a relatively simple topical method of pain relief in people suffering from neuropathic pain, for example, sympathetically mediated pain and peripheral neuropathy, and post-herpetic neuralgia.

Another object of this invention is to provide a topical composition of essential aromatic oils that will successfully allow a clinician to diagnose neuropathic pain more rapidly than applying GOB itself to the pain of the skin area.

Another object of the invention is to provide a composition of essential aromatic oils that will result in pain relief for a period substantially greater than that achieved by application of GOB as the sole agent and be self-administered.

An additional object of the invention is to provide a composition of essential aromatic oils that will result in a temporary or permanent reduction or elimination of the facial paralysis known as Bell's palsy and that this composition can be either orally or topically self-administered and have minimal side effects.

A further object of this invention is to provide a composition of essential aromatic oils that will substantially temporarily eliminate the pain from burns, including sunburns, relieve pain and itching from insect bites and can be self-administered and applied to the skin topically.

Another object of this invention is to provide a composition of essential aromatic oils that will result in temporary partial or complete elimination of pain in subjects suffering from neuropathic pain such as sciatica or post-herpetic neuralgia or sympathetically maintained pain and this composition can be administered orally.

A yet further object of this invention is to provide a composition of essential aromatic oils which will substantially temporarily eliminate sore throat associated with viral pharyngitis. This composition will be gargled for less than one minute and have minimal side effects.

Still further objects and advantages of this invention will become apparent through a consideration of the ensuing description.

### DETAILED DESCRIPTION OF THE INVENTION

The present invention is an improvement of the methods and expansion of uses as stated in the previous United States Patent issued in 1993 entitled “Diagnosis and Treatment of Various Neuralgias” (Frome)—U.S. Pat. No. 5,260,313. Reference to that patent provides evidence as to the usefulness of geranium oil bourbon for a range of conditions. The present invention has demonstrated an enhanced effect of the GOB through the addition of additional essential aromatic oils which act synergistically with the geranium oil bourbon. Through a trial and error process of screening, most known essential aromatic oils by themselves and in combination with geranium oil bourbon were clinically tested to determine which of these oils, if any, had a synergistic effect with the GOB.

I. Screening of Essential Oils

After treating over two thousand patients with topical geranium oil bourbon (Pelargonium graveolens) as the sole agent (subsequent to filing U.S. Pat. No. 5,260,313), most known essential oils were added individually to geranium oil bourbon, and the combined geranium oil bourbon plus single essential aromatic oil effectiveness for pain relief was tested. It was determined that five essential aromatic oils, when added individually to the geranium oil bourbon, increased the geranium oil bourbon effectiveness in relieving neuropathic pain when applied topically and could be said to act synergistically with geranium oil bourbon. The therapeutic effect, when combined, was greater than when each was administered individually.

The five additional essential aromatic oils that increased effectiveness and acted synergistically when added individually to the geranium oil bourbon in various concentrations were:

a) Lavender

b) Bergamot

c) Blue Chamomile

d) Eucalyptus

e) Tea Tree

All other known essential aromatic oils were individually determined to have no discernible effect when mixed with the GOB and topically did not result in pain relief.

Increased effectiveness is defined as increased speed of onset of neuropathic pain relief (NPR) and/or increased percentage of NPR and/or longer duration of NPR.

Study One: Topical Treatment of Neuropathic Pain

The purpose of this study was to evaluate and compare the efficacy for neuropathic pain relief in the application of eight topical compositions prepared singly or in combination for the relief of neuropathic pain. This was performed by utilizing only the five additional essential aromatic oils that had been determined to be effective in the screening process.

The study size was a total of forty volunteer subjects: 25 female and 15 male volunteer subjects. Their ages ranged from 18 to 80 years of age with a mean of 55. All subjects were previously diagnosed with moderate to severe neuropathic pain.

The method used on the forty subjects tested was simple trial and error. First, the effectiveness for neuropathic pain relief of geranium oil bourbon as the sole agent was tested on the subject. After pain relief was no longer present and on the next day, the geranium oil bourbon with one of the additional essential aromatic oils was applied and neuropathic pain relief effectiveness was evaluated.

The different therapeutically active agents in this Study One were as follows:

1) Geranium Oil Bourbon (GOB)

2) Lavender plus GOB

3) Lavender

4) Bergamot plus GOB

5) Bergamot

6) Blue Chamomile

7) Blue Chamomile plus GOB

8) Eucalyptus

9) Eucalyptus plus GOB

10) Tea Tree Oil

11) Tea Tree Oil plus GOB

12) GOB plus Lavender plus Bergamot plus Blue Chamomile plus Eucalyptus plus Tea Tree Oil.

A different composition was topically applied each treatment day to the area of maximum pain. The patients were asked to indicate the level of their pain immediately prior to the application of the compositions and then at intervals of 5, 15, 60 minutes, 2 hours, 4 hours, 8 hours, 24 hours, 3 days, 1 week, 1 month and 3 months. If the pain returned prior to the expiration of a time period the study for that patient was completed. If the pain was eliminated after 3 months, it was concluded that the pain relief was permanent.

Pain intensity was measured using the following Pain Intensity Scale:

Pain Intensity Scale

| | |
|---|---|
| 2.0 | Extremely Intense |
| 1.8 | Very Intense |
| 1.6 | Intense |
| 1.4 | Strong |
| 1.3 | Slightly Intense |
| 1.1 | Barely Strong |
| 1.0 | Moderate |
| 0.8 | Mild |
| 0.6 | Very Mild |
| 0.4 | Weak |
| 0.2 | Very Weak |
| 0.1 | Faint |
| 0 | No Pain Sensation |

Each patient was asked to choose that word that best described how the pain felt. The number corresponding to that word was then used to tabulate the intensity of the pain and to interpret the results. Significant pain relief was defined as >50% pain relief sustained for a specific period of time.

Results:

Each of the five additional essential aromatic oils (excluding GOB), when used individually, did not offer pain relief and were judged ineffective (not significant pain relief) on their own. Each of the five additional aromatic essential oils when combined individually with the GOB improved the pain relief achieved by using the GOB as the sole agent and offered significant pain relief, demonstrating that they acted synergistically with the GOB. The improvements in pain relief were greatest with lavender and blue chamomile.

Thus, one embodiment of the present invention consists of a composition comprising geranium oil bourbon with at least one other of the additional essential aromatic oils listed above, in any concentration. This embodiment could reasonably extend to use of fractions or purifications of each of the listed additional essential aromatic oils which may contain the active portions for analgesia. This composition can be used alone or in a composition with a pharmaceutically acceptable carrier in a topical form. Tests were performed on five subjects with previously diagnosed somatic pain (arthritis) and there was no pain relief reported with any of the combined formulas or the individual additional essential aromatic oils.

II. Combination of Essential Aromatic Oils

The next step in developing this invention was to combine each of the individual essential aromatic oils that were effective for neuropathic pain relief together with the geranium oil bourbon in various concentrations. This was again a trial and error method that lasted over a two-year period with approximately five hundred applications. The results indicated that when more than one of the additional essential aromatic oils listed above was added to the geranium oil bourbon, enhanced neuropathic pain relief effect was demonstrated compared to the addition of a single essential aromatic oil listed above.

While any combination of adding more than one of the five essential aromatic oils listed above was more effective than adding a single additional essential aromatic oil to GOB, adding all five additional essential aromatic oils in various ratios was the most effective. The most effective mixture (ratios) for neuropathic pain relief was determined by trial and error to be the following composition (“Composition”) of ingredients consisting of 20 parts, but was not limited to this specific 20-part composition:

| | | |
|---|---|---|
| a) | Lavender | 4 parts |
| b) | Bergamot | 2 parts |
| c) | Blue Chamomile | 2 parts |
| d) | Eucalyptus | 2 parts |
| e) | Tea Tree Oil | 2 parts |
| f) | Geranium Oil Bourbon | 8 parts |

Thus, a most preferred embodiment of the present invention consists of a 20-part Composition comprising geranium oil bourbon with all the other additional essential aromatic oils listed above, in the ratios indicated, but not limited to these ratios, as shown above. This embodiment could reasonably extend to use of fractions or purifications of each of the listed additional essential aromatic oils which may contain the active portions for analgesia. This Composition can be used alone or in a Composition with a pharmaceutically acceptable carrier in topical form.

Furthermore, this Composition provides a method for distinguishing nerve pain (neuropathic pain) from other types of pain. When administered topically, this Composition will completely or substantially eliminate chronic neuropathic pain within minutes for various durations of time. If the pain is not neuropathic in origin, pain relief will not occur.

Any of the compositions of the additional essential aromatic oils plus the GOB or the 20-part Composition can be distributed in a pharmaceutically acceptable carrier, known in the art as emulsions, solutions or suspensions including lotions, creams or ointments. These carriers can contain volatile diluents, such as alcohol (e.g. isopropyl alcohol) or glycol, as well as wetting, emulsifying and suspending agents.

In the forty subjects, the overall improvement in effectiveness for neuropathic pain relief of the topical composition containing GOB and the five additional essential aromatic oils in the 20-part mixture as stated above compared to the topical geranium oil bourbon as the sole agent ingredient was as follows:

| | | Composition (GOB plus |
|---|---|---|
| | GOB | 5 additional essential |
| | (sole ingredient) | aromatic oils) |
| Mean Duration of Pain | 2 to 24 hours | 2 hours to 3 months |
| Relief | | |
| Mean Onset of Pain Relief | Minutes | Few seconds to |
| | | minutes |
| Mean Reduction in Pain | 80-100% | 90-100% |
| (as Measured on Pain | | |
| Relief Scale) | | |
| Elimination of Swelling and | Minimal | 50-100% |
| Redness | | |

The geranium oil bourbon, used alone or in the composition including the additional essential aromatic oils, with or without a pharmaceutically acceptable carrier, is topically applied in therapeutically effective amounts to areas exhibiting the symptoms of neuropathic pain. Dosage amounts depend on how large are the patient's affected areas. Generally, one to ten drops are used; one drop for a smaller affected area and ten drops to larger affected areas. The topical composition containing the essential aromatic oil is spread over the complete area affected by pain and left to be absorbed by the skin.

Study Two: Oral Administration of 20-part Composition

In order to test for oral toxicity of the various compositions and related 20-part Compositions of oils as described above animal testing was conducted initially. After adding the various compositions, including the 20-part Composition to animals' (three dogs, six mice) food or water, a standard complete panel of blood tests was performed on the animals. No abnormalities were found in the blood tests of these animals fed dosages that exceeded one hundred times the dosage recommended for use in humans with any of the compositions. Furthermore, the animals exhibited no signs of side effects or toxicity.

In five subjects, with regard to pain from diabetic neuropathy, reflex sympathetic dystrophy, tic doloreux, post-herpetic neuralgia and sciatica, when one to two drops of the composition were mixed in 15 cc of water, juice or food and then swallowed, the pain from these conditions was eliminated within five to ten minutes. The duration of pain relief was variable. With regard to diabetic neuropathy, the pain relief often lasted as long as weeks to months. With regard to sciatica, tic doloreux and reflex sympathetic dystrophy, the pain relief was instantaneous and lasted from one to three hours.

Study Three: Sore Throat

In two subjects, with regard to pain relief from viral pharyngitis sore throat, when the 20-part Composition was added to 15 cc of juice or water and gargled for 20 to 30 seconds then repeated, the pain from the sore throat was substantially eliminated within two to three minutes. Pain relief lasted for approximately six hours.

Study Four: Burns and Insect Bites

In four subjects, when applied topically, the 20-part Composition temporarily (one to six hours) substantially eliminated pain and/or itching due to insect bites (bee stings or mosquito bites), or pain due to bums to the skin, including sunburn. Onset of relief was virtually instantaneous.

Study Five: Bell's Palsy

In two subjects, both with facial nerve palsy for four weeks, when swallowed, the 20-part Composition diluted in 15 cc of water or juice, substantially and permanently eliminated the paresis as a result of Bell's palsy within 24 hours. There is no other known effective treatment for Bell's palsy as of this date. When applied topically to the affected side of the face in two additional Bell's palsy subjects, the paresis was substantially eliminated within two hours but returned in a milder form within 24 hours. After repeat applications, the results were similar.

Study Six: Diagnosis

The 20-part Composition was applied topically in ten patients with previously diagnosed non-neuropathic pain (somatic pain) and as in earlier studies with GOB, pain relief did not occur, demonstrating no effect. Because of the effectiveness of the various compositions of the listed essential aromatic oils in treating particular neuropathies, the use of these compositions is also diagnostic for those neuropathies. This is beneficial as it provides the opportunity not only to treat the symptoms using these compositions, but also potentially to prevent or treat the cause of the pain. Additionally accurate diagnosis of neuropathy allows the medical practitioner to terminate treatments which are specific for different diseases but which were being used in an effort to relieve pain. Likewise, a diagnosis that rules out certain neuropathies due to the patient's lack of response to these compositions will allow the medical practitioner to pursue other treatments.

Geranium oil bourbon is specifically diagnostic for neuropathies including post-herpetic neuralgia and RSD, as described in U.S. Pat. No. 5,260,313. These two examples of neuropathies can then be distinguished from each other by their characteristics which have previously been found to be associated with one or the other. For example, a patient with post-herpetic neuralgia would have a history of infection by zoster virus infection and a recent rash at the site of the neuralgia while the peripheral neuropathy patient would usually have pain in the legs and feet.

By extension, diagnosis can also be performed not just by applying the geranium oil bourbon as described previously, but also by applying compositions of essential aromatic oils as described herein or the 20-part Composition and observing the presence or absence of relief from pain. The reaction is relatively quick (always within five minutes of application), and distinguishable, as the patient is acutely aware of the pain relief. From 80-100% of a patient's pain is relieved if the cause of the pain is neuropathic, while no relief is provided for somatic or visceral pain or pain that is not due to nerve injury. Burns, insect bites and sore throat are exceptions to this rule for diagnostic testing.

In all six studies, the method for determining pain relief was the same as in Study One, utilizing the Pain Relief Scale as described above.

Accordingly, it can be seen that we have provided improved compositions for treatment and diagnosis of neuropathic pain when compared to that presented in the Applicant's U.S. Pat. No. 5,620,313 issued in 1993 entitled “Diagnosis and Treatment of Various Neuralgias” (Frome). These improved compositions are effective in patients of either sex and utilize multiple therapeutically active agents either alone or prepared in suitable bases then applied periodically to the skin areas affected by such pain.

A composition of the essential aromatic oils can also be administered orally and is effective via that route for pain relief in post-herpetic neuralgia, sciatica and sympathetically maintained pain. The composition is also effective for relief of pain of burns and itching and pain of insect bites when applied topically. Furthermore, the improved composition is effective and safe in both oral and topical routes in the treatment of Bell's palsy.

Although each of the five additional individual essential aromatic oils, when mixed with the GOB, enhanced the therapeutic effect of the GOB, when all five were mixed together with the GOB in the above stated combination consisting of approximately but not limited to 20 parts, the therapeutic and diagnostic efficacy was maximized.

The 20-part Composition, or variations thereof, provides rapid onset of significant pain relief which lasts significantly greater than the GOB alone and provides significantly greater pain relief. Furthermore, the 20-part Composition has expanded uses over the GOB alone in that it is effective in treatment of burns and insect bites and Bell's palsy and when administered orally, it is effective in sciatica, peripheral neuropathy, tic doloreux, reflex sympathetic dystrophy, post-herpetic neuralgia and Bell's palsy.

Although the description above contains specific examples of the 20-part Composition used as the most effective concentration, it was also found in clinical trials to be safe and effective when mixed with one or more of the stated additional essential aromatic oils with GOB in different ratios than described for the 20-part Composition. Hence, use of any one of the stated additional essential aromatic oils, or combinations thereof, in any concentration, when added to GOB will be determined to be effective and within the scope of this invention.

Although the description above contains many specifics, these should not be construed as limiting the scope of the invention but as merely providing illustrations of some of the presently preferred embodiments of this invention. Various other embodiments and ramifications are possible within its scope.

Thus the scope of the invention should be determined by the appended claims and their legal equivalents, rather than by those presented in the Studies or by the examples given.

I claim:

1. A composition of geranium oil bourbon, with or without a pharmaceutically acceptable carrier, consisting of at least one of the following additional essential aromatic oils in any concentration or ratio for use in diagnosis or treatment of neuropathic pain and related conditions
a) Lavender
b) Bergamot
a) Blue Chamomile
b) Eucalyptus
e) Tea Tree

2. According to claim 1, a composition of geranium oil bourbon and consisting of more than one of the indicated additional essential aromatic oils in any concentration or ratio.

3. According to claim 2, a composition of geranium oil bourbon and consisting of all of the indicated additional essential aromatic oils in a concentration or ratio consisting of approximately but not limited to 20 parts as follows:

| | | |
|---|---|---|
| a) | Lavender | 4 parts |
| b) | Bergamot | 2 parts |
| c) | Blue Chamomile | 2 parts |
| d) | Eucalyptus | 2 parts |
| e) | Tea Tree Oil | 2 parts |
| f) | Geranium Oil Bourbon | 8 parts |

4. According to claim 1, a composition of geranium oil bourbon and consisting of at least one other essential aromatic oil of claim 1 in topical or oral dosage form.

5. A composition of geranium oil bourbon, with or without a pharmaceutically acceptable carrier, in oral dosage form and consisting of at least one of the following additional essential aromatic oils in any concentration or ratio for use in the treatment and relief of pain of viral pharyngitis accompanied by sore throat:
a) Lavender
b) Berganot
c) Blue Chamomile
d) Eucalyptus
e) Tea Tree

6. A composition of geranium oil bourbon, with or without a pharmaceutically acceptable carrier, in topical dosage form, consisting of at least one of the following additional essential aromatic oils in any concentration or ratio for use in the treatment of pain or itching due to insect bites and burns to the skin, including sunburn:
a) Lavender
b) Bergamot
c) Blue Chamomile
d) Eucalyptus
e) Tea Tree

7. A composition of geranium oil bourbon, with or without a pharmaceutically acceptable carrier, in topical or oral dosage form, consisting of at least one of the following additional essential aromatic oils in any concentration or ratio for use in the treatment of facial paralysis due to Bell's palsy:
a) Lavender
b) Bergamot
c) Blue Chamomile
d) Eucalyptus
e) Tea Tree

8. A method of treating the pain of post-herpetic neuralgia, reflex sympathetic dystrophy, peripheral neuropathy, trigeminal neuralgia, sciatica, tic doloreux, or other neuropathic pain conditions in patients by topically applying to skin areas affected with said pain or orally administering a therapeutically effective amount of a composition of geranium oil bourbon, with or without a pharmaceutically acceptable carrier, consisting of at least one of the following additional essential aromatic oils in any concentration or ratio:
a) Lavender
b) Bergamot
c) Blue Chamomile
d) Eucalyptus
e) Tea Tree

9. According to claim 8, a method of topically applying a composition of geranium oil bourbon, with or without a pharmaceutically acceptable carrier, consisting of all of the indicated additional essential aromatic oils in a concentration or ratio consisting of approximately but not limited to 20 parts:

| | | |
|---|---|---|
| a) | Lavender | 4 parts |
| b) | Bergamot | 2 parts |
| c) | Blue Chamomile | 2 parts |
| d) | Eucalyptus | 2 parts |
| e) | Tea Tree | 2 parts |
| f) | Geranium Oil Bourbon | 8 parts |

10. A method of treating sore throat due to viral pharyngitis by gargling a therapeutically effective amount of a composition of geranium oil bourbon consisting of at least one of the following additional essential aromatic oils in any concentration or ratio:
a) Lavender
b) Bergamot
c) Blue Chamomile
d) Eucalyptus
e) Tea Tree

11. According to claim 10, a method comprising gargling a composition of geranium oil bourbon, with or without a pharmaceutically acceptable carrier, consisting of all of the indicated additional essential aromatic oils in a concentration or ratio consisting of approximately but not limited to 20 parts:

| | | |
|---|---|---|
| a) | Lavender | 4 parts |
| b) | Bergamot | 2 parts |
| c) | Blue Chamomile | 2 parts |
| d) | Eucalyptus | 2 parts |
| e) | Tea Tree Oil | 2 parts |
| f) | Geranium Oil Bourbon | 8 parts |

12. A method of treating pain and itching due to insect bites and burns to the skin, including sunburn, in patients by topically applying a therapeutically effective amount of a composition of geranium oil bourbon consisting of at least one of the following additional essential aromatic oils in any concentration or ratio:
a) Lavender
b) Bergamot
c) Blue Chamomile
d) Eucalyptus
e) Tea Tree

13. According to claim 12, a method comprising topically applying a composition of geranium oil bourbon, with or without a pharmaceutically acceptable carrier, consisting of all of the indicated additional essential aromatic oils in a concentration or ratio consisting of approximately but not limited to 20 parts:

| | | |
|---|---|---|
| a) | Lavender | 4 parts |
| b) | Bergamot | 2 parts |
| c) | Blue Chamomile | 2 parts |
| d) | Eucalyptus | 2 parts |
| e) | Tea Tree Oil | 2 parts |
| f) | Geranium Oil Bourbon | 8 parts |

14. A method of treating Bell's palsy in patients by topically applying or orally administering a therapeutically effective amount of a composition of geranium oil bourbon consisting of at least one of the following additional essential aromatic oils in any concentration or ratio:
a) Lavender
b) Bergamot
c) Blue Chamomile
d) Eucalyptus
e) Tea Tree

15. According to claim 14, a method comprising topically applying or orally administering a composition of geranium oil bourbon, with or without a pharmaceutically acceptable carrier, consisting of all of the indicated additional essential aromatic oils in a concentration or ratio consisting of approximately but not limited to 20 parts:

| | | |
|---|---|---|
| a) | Lavender | 4 parts |
| b) | Bergamot | 2 parts |
| c) | Blue Chamomile | 2 parts |
| d) | Eucalyptus | 2 parts |
| e) | Tea Tree Oil | 2 parts |
| f) | Geranium Oil Bourbon | 8 parts |

16. A method of diagnosing the pain of post-herpetic neuralgia, reflex sympathetic dystrophy, trigeminal neuralgia or other neuropathic pain conditions in patients comprising topically applying a diagnostically effective amount of a geranium oil bourbon composition to skin areas affected with said pain, and observing a significant decrease in said pain within minutes, said geranium oil bourbon composition consisting of at least one of the following additional essential aromatic oils in any concentration or ratio:
a) Lavender
b) Bergamot
c) Blue Chamomile
d) Eucalyptus
e) Tea Tree

17. The method of claim 16 wherein the geranium oil bourbon composition is present in a pharmaceutically acceptable carrier.

18. The method of claim 16 wherein the geranium oil bourbon composition is left on the skin to be absorbed into the skin of the affected area.

19. The method of claim 16 wherein the reduction of pain is further associated with a reduction in redness (if present) of the affected skin area.

20. The method of claim 16 wherein the reduction of pain is further associated with a reduction in swelling (if present) of the affected skin area.
